(19) United States
(12) Patent Application Publication
(10) Pub. No.: US 2011/0039267 A1
(43) Pub. Date: Feb. 17, 2011

(54) Sample Preparation And Detection Of Analytes Using Silica Beads

(76) Inventors: Cha-Mei Tang, Potomac, MD (US); Daniel Adams, Kensington, MD (US); Steingrimur Stefansson, Gaithersburg, MD (US)

Correspondence Address: Stanislav Torgovitsky;ROYLANCE, ABRAMS, BERDO & GOODMAN, L.L.P., Suite 600, 1300 19th Street, N.W., Washington, DC, 20036, US

(21) Appl. No.: 12/856,448

(22) Filed: Aug. 13, 2010

Related U.S. Application Data
(60) Provisional application No. 61/233,766, filed on Aug. 13, 2009.

(51) Int. Cl. C12Q 1/68; G01N 33/569; B01D 15/04

(52) U.S. Cl. 435/6; 435/7.37; 210/660; 210/263

(57) ABSTRACT

This invention provides methods for concentration of analyte from biological sample, or various liquid samples and methods for identification of the analyte. The method includes providing silica beads conjugated with analyte recognition element for concentration of analyte from sample, packaging the beads into a cartridge, and attaching the cartridge to a sample holder. After the analyte concentration, additional steps can be implemented to identify the analyte to provide visual positive/negative, qualitative or quantitative results. Another exemplary embodiment of the present invention provides a system for concentration of analyte from biological sample or other various liquid samples using silica beads. According to an exemplary implementation, the system includes a cartridge comprising silica beads conjugated with analyte recognition element to concentrate at least one analyte from the sample, and a sample holder having the cartridge attached thereto. A sample is passed through the cartridge.

This application claims benefit under 35 U.S.C. §119 from U.S. Provisional Application No. 61/233,766, filed on Aug. 13, 2009, the entire content of which is incorporated herein by reference.

### FIELD OF THE INVENTION

The invention is generally in the field of analytes detection, and provides methods, apparatus and reagents for concentration of analyte from blood, serum, urine, saliva, cerebral spinal fluid, other body fluids, water, liquids, growth media, etc. using silica beads. The analytes can be proteins, hormones, DNA, bacteria, fungus, viruses, etc. The format of the detection after sample preparation can vary. An exemplary implementation of certain embodiments of the present invention provides for detection of bacteria for diagnosis of sepsis from blood or blood pikes in growth media.

### DESCRIPTION OF RELATED ART

Conventional methods for detecting analytes from samples are described below.

The term “analyte” refers to any substance or chemical constituent that is subject to analysis.

As used herein, the term “sample” is intended to mean predominantly a liquid to be tested. The sample could contain the analyte of interest.

The term “analyte recognition element” refers to a binding moiety that recognizes the analyte and binds to the analyte. The analyte recognition element can be coated or elementally bound to beads in a column. The recognition element is bindingly receptive towards the analyte.

A specific example of an application of methods for detecting analytes is detection of low concentration of pathogens in blood. Conventional diagnosis of bacteremia and sepsis are described as follows.

The diagnosis of sepsis begins with a patient questionnaire followed by a clinical and laboratory screening. Diagnosis of sepsis begins upon a patient presenting a number of symptoms, including high fever, hypotension, tachypnea, shaking, chills, weakness, confusion, nausea, vomiting, diarrhea, etc. In an initial clinical screening, a physical analysis of the patient may include a physician testing, vital signs (temperature, blood pressure, respiratory rate), infection entry points (cuts, wounds, intravenous lines, catheters), secondary infections (abscesses, thrush, rashes, necrotizing tissue, etc.), a cardiac exam (murmurs, hypotension, hypertension), and/or a pulmonary exam (impaired oxygenation, respiratory distress, secondary infections). Initial laboratory screening for patients presenting with sepsis will include blood, urine and mucus cultures. Further testing will involve blood counts, urinalysis, blood urea nitrogen, glucose, electrolyte, creatinine, lactose dehydrogenase, amylase, etc.

Though culturing is the “gold standard” for diagnosis of bacteremia and sepsis, the method is both expensive and more importantly, time-consuming. A typical culture takes a minimum of 24 hours for initial results, and up to 7 days for full species identification. This may include multiple sub-cultures and the need of specialists, including a microbiologist, hematologist or expensive equipment, to identify the pathogen present. Furthermore, culturing has high incidences of false positives and false negatives.

A number of alternative methods for determining the presence of septic pathogens are available, many being FDA approved, or soon to be FDA approved. These technologies include biomarkers, nucleic acid probes, specialized substrates, specialized culture media, ELISA, lateral flow, etc. Each of these methods has their specific attributes, and limitations; however a common limitation with the majority of these methods is the lack of sensitivity and therefore the need for a culture step. Below is a list of the more prevalent conventional methods available for diagnostic use along with their advantages and limitations.

PCR Based Methods. PCR based detection strategies are capable of rapidly determining the specific bacteria at low concentrations (theoretically a single cell). However these methods utilize only small samples size of a few micro liters. To collect and concentrate low concentration of analyte from sample is a challenge.

PNA FISH™ Methods. Peptide nucleic acid probes (PNA-FISH™) is a method in which a specific fluorescent probe hybridizes to a segment of rRNA, in vivo, allowing for the visual detection and species identification. PNA FISH™ testing limitations are similar to cell culture and PCR. The process involves an initial culture phase of at least 8 hours followed by a 1.5-2.5 hour assay protocol and subsequent verification by microscopic analysis, meaning the whole procedure takes more than 10 hours.

Lateral Flow Assay Methods. Also known as ticket assays, are usually antibody based immunoassays with a colorimetric indicator. In a typical assay, a sample is added to the end of a solid support. As capillary action draws the sample along the solid support it reacts with substrates already pretreated in specific zones on the support. A color change usually indicates the presence of the antigen. This method can be highly specific if there is an antibody with an appropriate antigenic marker recognition capability. This method is very rapid taking about 20-30 minutes. However, it requires a small sample (less than 1 mL) which often decreases sensitivity. Samples must have high concentrations of cells, so culturing is usually needed before the test can be used, or concentration method is required.

Substrate Defined Methods. Are methods in which a substrate, reactive to a specific organism or pathogenic antigen, reacts upon binding to said antigen. This method can often be quantified, or qualified, by colorimetric or fluorescent detection systems.

### SUMMARY OF THE INVENTION

An exemplary embodiment of the present invention provides a method for concentration of analyte from biological sample, such as blood, serum, urine, saliva, cerebral spinal fluid or other body fluids, or other types of liquid samples, such as water, juice, milk, spiked growth media, etc., using silica beads. According to an exemplary implementation, the method includes providing silica beads conjugated with analyte recognition element for concentration of analyte from sample, packaging the beads into a cartridge, and attaching the cartridge to a sample holder. A sample is passed through the cartridge.

Another exemplary embodiment of the present invention provides a system for concentration of analyte from biological sample, such as blood, serum, urine, saliva, cerebral spinal fluid or other body fluids, using silica beads. According to an exemplary implementation, the system includes a cartridge comprising silica beads conjugated with analyte recognition element to concentrate at least one analyte from the sample, and a sample holder having the cartridge attached thereto. A sample is passed through the cartridge.

Yet another exemplary embodiment of the present invention provides a method for capture and identifies analyte from biological sample, such as blood, serum, urine, saliva, cerebral spinal fluid or other body fluids, or other types of liquid samples, such as water, juice, milk, spiked growth media, etc., using silica beads. According to an exemplary implementation, the method includes conjugating silica beads with analyte recognition element to capturing analyte from sample, packaging the beads in a cartridge; connecting the cartridge to a sample holder, passing a sample through the cartridge, and adding a regent after analyte capture to provide visual identification of the detected analyte.

### BRIEF DESCRIPTION OF THE DRAWINGS

The above and other exemplary features, aspects, and advantages of the present invention will be more clearly understood from the following detailed description taken in conjunction with the accompanying drawings, in which like reference numerals will be understood to refer to like parts, components and structures, where:

FIG. 1. Illustrates an example of 0.1 mm diameter silica particles.

FIG. 2. Illustrates an exemplary setup of an exemplary implementation of a test format including a silica bead filled cartridge attached to a syringe according to an exemplary embodiment of the present invention.

FIG. 3. Provides a schematic diagram of the analyte concentration cartridge according to an exemplary embodiment of the represent invention where: a sample flows in through the top; the antibody coated beads capture pathogens and retain it in a specific area in the cartridge; the analyte of interest are S. aureus and E. coli bacteria; the sample is blood; and the analyte recognition elements are antibodies.

FIG. 4. Provides a schematic diagram of another exemplary embodiment of the present invention comprising another analyte concentration cartridge to capture analyte.

FIG. 5. Illustrates yet another exemplary embodiment of the present invention including two cartridges layered with silica conjugated antibodies: E. coli O157 and E. coli O26. (−) The negative control i.e. no cells, only antibodies in buffer; (+) both cell types flowed through the cartridge.

FIG. 6. Illustrates yet another exemplary embodiment of the present invention including cartridge exposed to only E coli O26 cells without O157 cells.

FIG. 7. Illustrates yet another exemplary embodiment of the present invention including capture and detection of E. coli O157:H7 using 0.1 mm silica particles in a cartridge where silica beads are coated with monoclonal O157:H7 for capture and Cy5 conjugated E. coli O157 to detect presence of cells, and the fluorescence was detected using a fluorometer.

### DETAILED DESCRIPTION OF EXEMPLARY EMBODIMENTS

Several embodiments of the present invention will now be described in detail with reference to the annexed drawings. As noted above, in the drawings, the same or similar elements are denoted by the same reference numerals even though they are depicted in different drawings. In the following description, detailed descriptions of known functions and configurations incorporated herein have been omitted for conciseness and clarity. Likewise, certain naming conventions, labels and terms as used in the context of the present disclosure are, as would be understood by skilled artisans, non-limiting and provided only for illustrative purposes to facilitate understanding of certain exemplary implementations of the embodiments of the present invention.

Blood, serum, urine, saliva, cerebral spinal fluid and other body fluids are complex matrices. The concentration of analytes becomes difficult when the concentration of the analytes is low, so the signal (optical or electrical) generated by the detection method is not sufficient to produce an answer. Another application is to detect pathogens in water samples, such as juices, milk, produce wash, beach water, etc. Other times, the method to detect the low concentration requires a long time or needs expensive instrumentation.

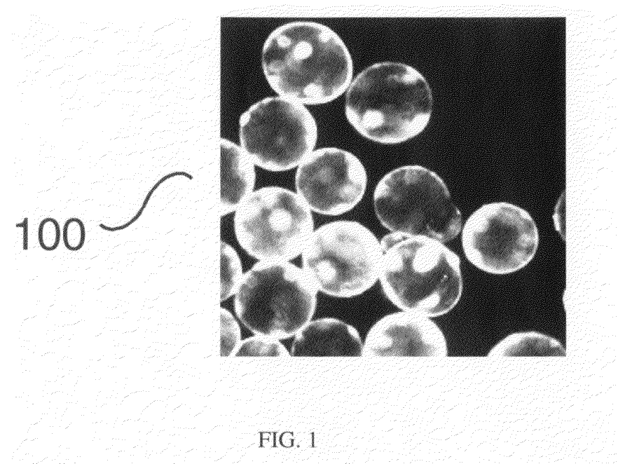

There is a need to detect low concentration of analytes in large sample volumes, in short periods. For detection of pathogens, it is common practice to place the sample in growth media and grow the analyte in media to stationary phase, taking 8 hours to a few days. Long growth time is required due to the difficulty of detecting analyte in low concentrations. To address such drawback, and without limitation, an exemplary embodiment of the present invention provides conjugated silica particles (or called silica beads, or glass beads) for sample preparation which is an alternative to Sepharose beads, plastic beads and magnetic beads. FIG. 1 shows a photo of silica particles (or beads) 100. Exemplary implementations of certain embodiment of the present invention are not complicated, the assay is rapid, and results can be reported as positive/negative, qualitative or quantitative.

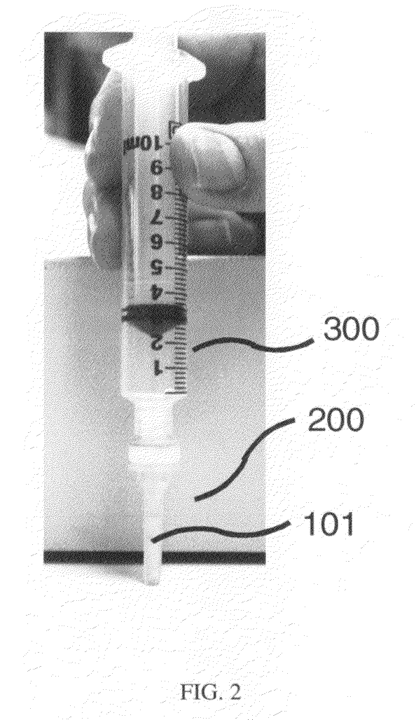

Another exemplary embodiment of the present invention provides an apparatus for performing the method noted above. In an exemplary implementation, silica particles coated with analyte recognition element are packed into a cartridge. The cartridge is attached to a sample holder tube. To pass the sample through the cartridge, a syringe, pump or vacuum suction can be used. FIG. 2 shows an exemplary configuration consisting of a cartridge 200 filled with beads 101 and syringe 300. The cartridge can have different shapes. The syringe can have different shapes and methods of operation. The amount of beads and sizes in the cartridge can vary.

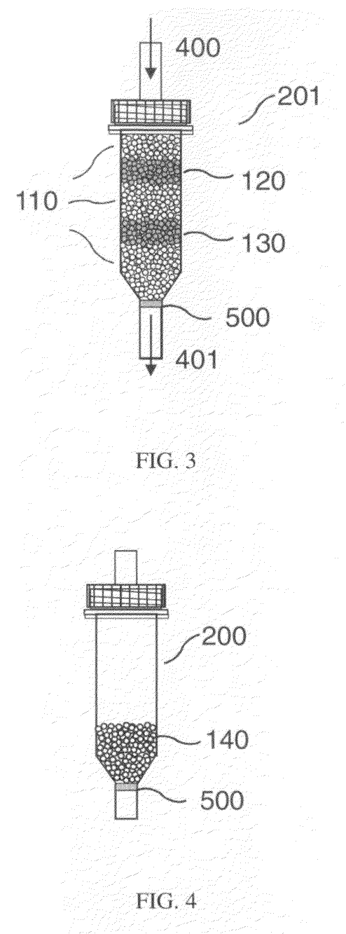

Much like column chromatography, according to an exemplary implementation, a cartridge can be filled with one or more layers of analyte capture silica particles (FIG. 3). FIG. 4 is another implementation of the glass filled cartridge. It is for detection of just one analyte. The cartridges in FIGS. 3 and 4 are different from the cartridge shown in FIG. 2. There can be many variations to implement the cartridges depending on the needs consistent with the embodiments of the present invention. For example, a feature such implementations should have in common are (1) fits or grid that can hold the beads and let sample through without clogging, (2) one or more inlets, (3) one or more outlets, (4) ability to connect to the sample container and (5) material for the cartridge which does not promote binding of proteins and analyte to its surface.

### METHODS

According to an exemplary implementation of certain embodiment of the present invention, a sample preparation method to detect analyte in sample is described as follows. In this illustration a cartridge is filled with silica particles coated with at least one capture analyte recognition element, shown in FIG. 3 or 4. Samples are passed through the cartridge. Analyte of interest from the sample will be capture on the silica particle. This is followed by washing.

The analyte includes proteins, bacteria, viruses, spores, oocysts, cells, cell fragments, receptors, nucleic acid, oligonucleotides, antibodies, enzymes, antibiotics, peptides, carbohydrates, hormones, toxins, disease markers, DNA, cDNA, miRNA, mRNA, RNA, natural organic compounds, synthetic organic compounds such as pesticides, pharmaceuticals, food additives, dyes, and inorganic compounds.

According to an exemplary implementation, the silica particles can be glass beads that contain silicon.

According to another exemplary implementation, the capture analyte recognition element can be antibodies, antibiotics, an antigen target for an antibody analyte, cell receptor protein, avidin, NeutrAvidin®, biotin, nuclear acid or related to nucleic acid (e.g., oligonucleotide, DNA, cDNA, microRNAs, mRNA and RNA), riboprobes, polysaccharide, monosaccharide, oligosaccharide, Poly-L-lysine, Polymyxin B, Daunomycin, Acridine, Spermine, aptamers, Vectabond™, amino-ccyl silane, Superfrost Plus™, Maple's, NaOH/Poly-L-lysine, bozymes, enzymes, ligands, cell and cell fragment as well as other biological particles.

According to yet another exemplary implementation, the sample can be blood, serum, urine, saliva, cerebral spinal fluid, other body fluids from people, animals, fish, and other living organisms. The sample can also be water, such as produce wash, water from beach, river, streams, irrigation duct, lakes, ponds, ocean, run-offs, etc., or other liquids, such as juice, milk, etc. The sample can also be growth media spiked with body fluids, food, water, juice, produce, or other materials.

According to yet another exemplary implementation, the cartridge can be filled with silica particles coated with one or more types of analyte recognition element. If there is more than one type of silica particle coatings to capture different analytes, the different silica particles can be mixed together or placed in layers as shown in FIG. 3. The sample flow into the cartridge 201 via 400 and leaves via 401. The cartridge 201 is filled with unfunctionalized silica particles 110 with layers of silica particle coated with two different types of capture analyte recognition elements, 120 and 130. The beads are held in the cartridge by a frit 500.

In an exemplary embodiment of the present invention, a prefilter with pores larger than silica particle size may be introduced to remove large particles from sample.

In another exemplary embodiment of the present invention, a method to move the sample through the cartridge can use syringe, pump or vacuum suction, by pushing or pulling the sample through the cartridge. To improve analyte capture, the sample can be pumped in and out more than one time through the cartridge or the sample can be pumped through the cartridge in a continuous format.

After the analyte is captured from the sample, exemplary embodiments of the present invention allow for various methods that can be used to identify or report the detection. For example, the detection method can be directly on the beads in the cartridge, or reported outside the cartridge.

According to an exemplary embodiment of the present invention, direct detection of the analytes on the beads in the cartridge can be rapid. If the analyte is a pathogen, chromogenic reagents can be used to visualize immobilized bacteria. The chromogenic reagents will directly react with bacteria to produce color change. Examples of chromogenic reagents including, Silkworm Larvae Plasma (SLP), Peptidoglycan binding proteins, Alkaline Phosphatase substrate, Peroxidase substrate, Peptidoglycan Recognition Protein for their ability to give a distinctive staining for easy positive/negative evaluation.

In an exemplary implementation, if beads are prepared to capture more than one pathogen and the beads 120 and 130 are layered, colored bands will indicate the presence and type of the pathogens, as shown in FIG. 3. The position of each layer can be marked on the outside of the cartridge for easy identification of the species present.

In an exemplary implementation, the analyte can be tagged with a detector analyte recognition element conjugated to a fluorescent label or HRP to form a sandwich, i.e. (capture analyte recognition element)—analyte—(detector analyte recognition element conjugated with fluorescent label or HRP). HRP can be used for visual detection.

In addition to visual detection, other method can be applied following sample preparation without departing from the scope of the present invention, and they could be ELISA, fluorescent plate readers, PCR, PNA FISH, plating, lateral flow assay, substrate defined methods, sandwich immunoassays, flow cytometry, mass spectrometry, etc.

In an exemplary implementation, for fluorescence detection, the detector analyte recognition element, the fluorescent label or the HRP are released from the beads. The solution is tested in a fluorometer for fluorescent labels, ELISA plate readers for HRP.

In another exemplary implementation, the report of detection of analyte is positive if the signal is greater than the threshold. One often used threshold condition is signal from zero concentration plus three standard deviations. Quantitative results can be obtained using fluorescence label, while HRP can provide qualitative results.

To perform genetic testing, cells need be lysed and DNA or RNA collected for PCR or isothermal amplification. The cells can be lysed while attached to the silica particles.

To perform lateral flow assay, the analyte has to be released from the silica particle column and the solution on a lateral flow test kit.

To perform plating of pathogens, the pathogens need to be released from the silica particles and the solution spread on a plate, or directly plating the silica particles.

To perform FISH or PNA FISH with the captured pathogens or cells on the silica beads, the steps are to perform FISH or PNA FISH on the silica particles followed by lysing the cells, extract the lysis solution from the cartridge, and read the fluorescence in a fluorometer.

To perform the detection of the analyte on an ELISA plate to obtain qualitative results, the steps are to perform a sandwich assay on the silica particle and followed the steps of ELISA, followed by extract the solution and placing it into an ELISA plate and read the result on a plate reader.

For pathogen detection a short enrichment step, not to saturation, can precede the pathogen concentration in the sample holder. Enrichment can also be performed directly in the syringe.

According to exemplary embodiments of the present invention, the passed through sample can still be used for other testing.

### Apparatus

According to another exemplary embodiment of the present invention, an apparatus is provided to allow the exemplary methods described above where one or more types of silica particles coated with capture analyte recognition element(s) are placed in a cartridge that sample can flow through using syringe, pump, or vacuum suction.

According to an exemplary implementation, the silica particles can be glass beads that contain silicon. The size of beads can be from a few tenths of microns to thousands of microns. They can be spherical or nearly spherical in shape or irregularly shaped. The amount of silica particles used can vary. For visualization of the silica particle. FIG. 1 is a photo of silica particle functionalized with NeutrAvidin and stained with biotin-FITC.

According to another exemplary implementation, the capture analyte recognition element can be antibodies, antibiotics, an antigen target for an antibody analyte, cell receptor protein, avidin, NeutrAvidin®, biotin, nuclear acid or related to nucleic acid (e.g., oligonucleotide, DNA, cDNA, microRNAs, mRNA and RNA), riboprobes, polysaccharide, monosaccharide, oligosaccharide, Poly-L-lysine, Polymyxin B, Daunomycin, Acridine, Spermine, aptamers, Vectabond™, amino-ccyl silane, Superfrost Plus™, Maple's, NaOH/Poly-L-lysine, bozymes, enzymes, ligands, cell and cell fragment as well as other biological particles.

According to yet another exemplary implementation, much like column chromatography, a cartridge can be filled with one or more layers of analyte capture silica particles (FIG. 3). FIG. 4 is another implementation of the silica particle filled cartridge 210 with just one type of silica particles 140. It is for detection of just one analyte.

FIG. 4 format is also suitable to fill the cartridge with a mixture of silica particles with more than one type of capture analyte recognition elements. It is for detection of more than one analyte.

According to exemplary embodiments of the present invention, cartridges in FIGS. 3 and 4 are different from the cartridge shown in FIG. 2. There can be many variations to implement the cartridges depending on the needs. The feature they need to have in common are (1) frits or grid that can hold the beads and let sample through without clogging, (2) one or more inlets, (3) one or more outlets, (4) it can be connected to the sample container and (5) the material for the cartridge does not promote binding of proteins and any analyte to its surface.

### EXAMPLES

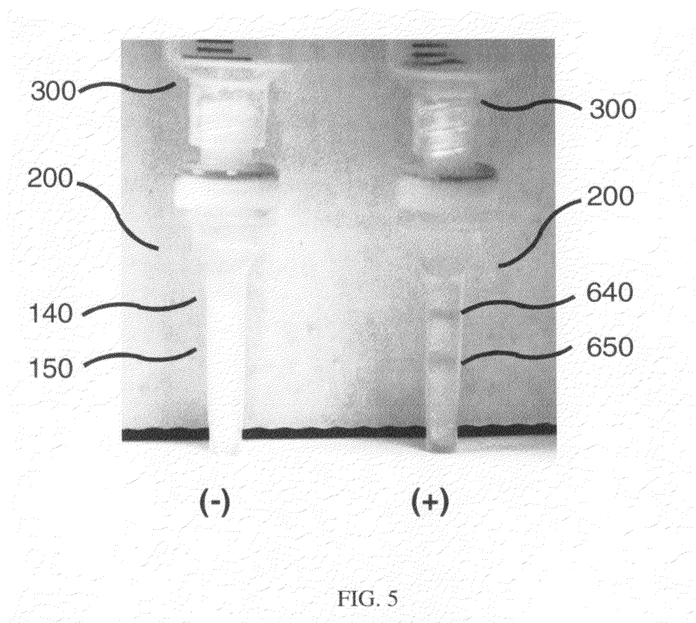

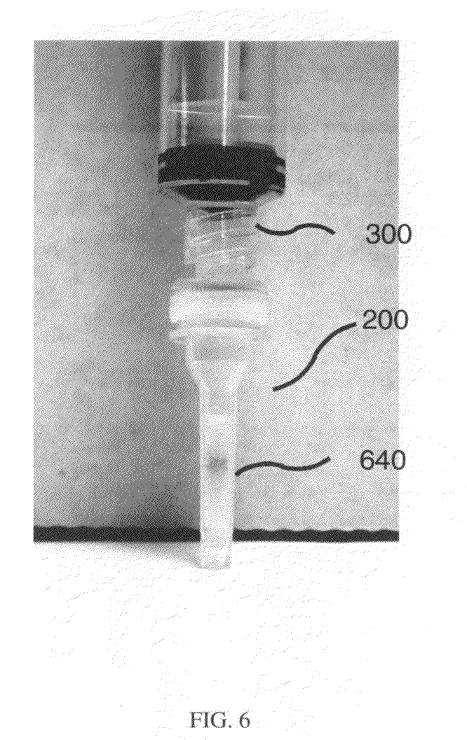

A detailed description of an exemplary method to detect two different E. coli is given below as a non-limiting example of certain embodiments of the present invention. Three cartridges were made as shown in FIGS. 5 and 6. In FIGS. 5 and 6, the apparatus consists of syringe 300, cartridge 200, and silica beads 140 and 150. Silica beads labeled with either antibody against E. coli O157:H7 or E. coli O26 were layer in the following manner: 250 mg uncoated beads on the bottom layer, 10 mg anti-026 silica, 250 mg uncoated beads, 10 mg anti-0157 silica and lastly a layer of 250 mg uncoated beads. Analytes are prepared for three experiments.
Experiment 1: As a negative control, antibodies against O26 and O157 were placed in 10 mL of buffer without cells.
Experiment 2: 10 mL of 10,000 cfu/mL of E. coli O157 cells with 10,000 cfu/mL of E. coli O26 cells were incubated with HRP labeled monoclonal antibody, against E. coli O157 and E. coli O26, for 30 minutes.
Experiment 3: 10 mL of 10,000 cfu/mL E. coli O26 cells were incubated with HRP labeled monoclonal antibody, against E. coli O26, for 30 minutes.

Experimental procedures are as follows. A 10 mL mixture of antibodies with no cells was passed over the first cartridge; the E. coli O157 and O26 sample was passed over the second cartridge, and the E. coli O26 sample was passed over the second cartridge. The cartridges were washed with 3 mL PBS each, approximately 1 minute. A diluted 1:10 in PBS HRP substrate (Pierce Thermo) was added to each cartridge and a picture was taken after 1 minute (FIGS. 5 and 6). As seen in FIG. 5 the cartridge not exposed to cells labeled (−) does not react (Experiment 1). The cartridge exposed to both E. coli strains reacts and turn blue bands for each strain, band 640 for HRP labeled E. coli O26 cells and band 650 for HRP labeled E. coli O157 cells in FIG. 5 labeled (+) (Experiment 2). The cartridge exposed to only O26 cells react by showing only one upper band 640 (FIG. 6) (Experiment 3).

Another example is for detection of more than one pathogens for sepsis. Prototype capture cartridges were prepared using a cartridges 200, a highly porous frit 500 and our silica particles layered as seen in FIG. 3. The 3-D layers isolated specific cells and a HRP visualization reagent was used to detect the presence of the cells. The example is a cartridge prepared to detect two different pathogens: Escherichia coli (E. coli) and Staphylococcus aureus (S. aureus). The capture and detector analyte recognition elements used are antibodies for E. coli and S. aureus. The cartridge preparation is shown in FIG. 3, for example band 120 for S. aureus and band 130 for E. coli. Blood from suspect sepsis patient is flow through the cartridge to capture E. coli and S. aureus.

Other assay methods to provide rapid visualization of diagnostic also exit. The assay can also be adapted to include an ATP Luciferease reagent where a camera or instrument can detect the presence of the light. Antibodies against cell surface markers can be used as the detection reagent. A mixture of reagents can be used. For instance a LAL test will verify the presence of Gram negative cells while an antibody can detect gram positive cells. An alternative method is using general antibodies against common cell surface markers. This can be done by adding HRP labeled antibodies reactive to gram positive and gram negative antigens.

Detection post analyte capture can take various formats.

For fluorescent/luminescent sandwich immunoassay, additional steps needed after analyte captures are incubation with fluorescent detection antibodies followed by wash. For high concentrations, the emission can be read by eye.

For low concentrations, the signal needs to be read by a fluorometer or luminometer. The additional following steps are needed: releasing the antibody from the beads and read the fluorescence in a fluorescent plate reader, fluorometer, or luminometer.

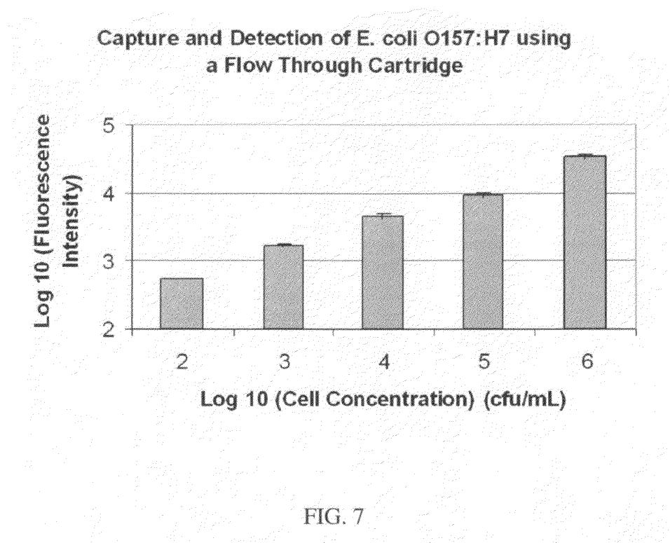

FIG. 7 shows the results of capture and detection of E. coli O157:H7 using 0.1 mm silica particles in a cartridge. Silica beads are coated with monoclonal E. coli O157:H7 for capture and Cy5 conjugated E. coli O157 to detect presence of cells. The fluorescence is read using spectrofluorometer, Signalyte™-II. The results are quantitative.

To perform DNA amplification following pathogen or cell capture, the pathogen or cell can be lyses while they are attached to the silica beads. There are a number of protocols for the extraction of nucleic acids from cells using silica beads. The following is a description using Bi0stic™Bacteremia DNA Isolation Kit (MO Bio Laboratories, Inc.) with a slightly modified protocol using the cartridge in place of MicroBead Tubes. The protocol, available at [http://www.mobio.com/files/protocol/12240-50.pdf], consists of the following
1. Add the “Cell Lysis/Inhibitor Solution” to the cartridge, heat and vortex sample to release DNA.
2. Remove the supernatant and add the “Inhibitor Solution” to the supernatant.
3. Add “DNA Binding Solution” to supernatant and add to a spin filter, DNA will bind to the filter.
4. Wash twice and add “Elution Solution”.
5. Elute DNA by spinning and run sample by PCR or other DNA amplification method.

Although exemplary embodiments of the present invention have been disclosed for illustrative purposes, those skilled in the art will appreciate that various modifications, additions, and substitutions are possible, without departing from the scope of the present invention. Therefore, the present invention is not limited to the above-described embodiments, but is defined by the following claims, along with their full scope of equivalents.

We claim:

1. A method for concentration of analyte from biological sample, such as blood, serum, urine, saliva, cerebral spinal fluid or other body fluids, using silica beads, the method comprising:
providing silica beads conjugated with analyte recognition element for concentration of analyte from sample;
packaging the beads into a cartridge; and
attaching the cartridge to a sample holder,
wherein a biological sample is passed through the cartridge.

2. A system for concentration of analyte from biological sample, such as blood, serum, urine, saliva, cerebral spinal fluid or other body fluids, using silica beads, the system comprising:
a cartridge comprising silica beads conjugated with analyte recognition element to concentrate at least one analyte from the sample; and
a sample holder having the cartridge attached thereto;
wherein a biological sample is passed through the cartridge.

3. A method for capture and identify analyte from biological sample, such as blood, serum, urine, saliva, cerebral spinal fluid or other body fluids, using silica beads, the method comprising:
conjugating silica beads with analyte recognition element to capturing analyte from sample;
packaging the beads in a cartridge;
connecting the cartridge to a sample holder;
passing a biological sample through the cartridge; and
adding a regent after analyte capture to provide visual identification of the detected analyte.
